(19) United States
(12) Patent Application Publication
(10) Pub. No.: US 2024/0423950 A1
(43) Pub. Date: Dec. 26, 2024

(54) 5-MAPB OR 6-APB FOR USE IN DRUG-ASSISTED PSYCHOTHERAPY

(71) Applicant: CLEARMIND MEDICINE INC., Tel Aviv (IL)

(72) Inventor: Ezekiel Golan, Vancouver (CA)

(73) Assignee: CLEARMIND MEDICINE INC., Tel Aviv (IL)

(21) Appl. No.: 18/699,185

(22) PCT Filed: Oct. 6, 2022
(86) PCT No.: PCT/IB2022/059561

§ 371 Date: Apr. 5, 2024

Related U.S. Application Data
(60) Provisional application No. 63/253,443, filed on Oct. 7, 2021.

(51) Int. Cl. A61K 31/343

(52) U.S. Cl. CPC A61K 31/343

(57) ABSTRACT

The present invention relates to methods of drug-assisted psychotherapy, particularly with methods employing 5-MAPB or 6-APB. The present invention also relates to kits comprising 5-MAPB or 6-APB for use in the treatment of subjects undergoing psychotherapeutic intervention. In particular, methods and kits for drug-assisted psychotherapy with 5-MAPB or 6-APB for a variety of disorders and/or conditions, including PTSD, GAD and relationship distress are provided.

### CROSS-REFERENCE TO RELATED APPLICATION

This application claims priority to and the benefit of U.S. Provisional Patent Application No. 63/253,443, filed Oct. 7, 2021 and titled “BENZOFURANS AS FAIL SAFES FOR MDMA THERAPY,” the entire content of which is incorporated herein by reference.

### FIELD OF THE INVENTION

The present invention relates to methods of drug-assisted psychotherapy, particularly to methods employing 1-(Benzofuran-5-yl)-N-methylpropan-2-amine (“5-MAPB”) or 1-Benzofuran-6-yl propan-2-amine (“6-APB”) for people for whom 3,4-methylenedioxy-methamphetamine (“MDMA”) therapy is not effective, desirable, or appropriate.

Traditional psychotherapy or counselling is relatively ineffective for the individuals most severely impacted by mental health issues or emotional health issues, and as a result the current “best practice” alternative to traditional therapy or counselling is typically prescribing a one or more of a wide range of psychiatric medications, which are often relatively ineffective and/or have unpleasant and even dangerous side effects.

Drug-assisted psychotherapy is a branch of psychiatry that brings together the benefits of drug and psychological treatments with the goal of synergically enhancing each other's effects. One aspect of this interaction appears to be that the drug treatment enhances the patients rapport with the therapist, increases flexibility in the patient's mind, allowing the patient to discover and incorporate new ways of thinking about their situations/dilemmas. Furthermore, compliance with therapy is a long standing and difficult problem, potentially limiting the efficacy of all interventions and frequently being the most limiting factor in providing sustained psychotherapeutic benefit. Combining pharmacotherapeutic with psychotherapeutic intervention can improve compliance with treatment plans. Yet further, treatments that reduce anxiety help prevent episodes of dissociation and panic that can derail therapy.

There are ongoing, as well as completed, clinical trials of MDMA-assisted psychotherapy (see, for example, NCT0009064; NTC02008396; NTC00353938; NTC01958593 and NCT01458327, NCT02427568) exploring the uses of MDMA in clinical treatment of, inter alia, post-traumatic stress disorder (“PTSD”) and anxiety.

One of the problems that therapists may encounter is that clients approach them who have had long exposures to MDMA through either recreational use or other contexts. With sufficient exposure to MDMA, the human brain adapts by developing tolerance, which results in either having a negative response or a neutral response to MDMA. Either of these responses is not helpful in the context of therapy. Both 5-MAPB or 6-APB could be used as a substitute for MDMA with individuals who have become tolerant to MDMA. 5-MAPB or 6-APB could also be used in therapy for individuals who, for any reason, MDMA-assisted therapy is not desirable, appropriate, or applicable.

### SUMMARY OF THE INVENTION

This invention is a method for treating a subject undergoing a psychotherapeutic intervention comprising administering to the subject 5-MAPB or 6-APB for whom MDMA-assisted psychotherapy has become ineffective. 5-MAPB or 6-APB are both similar enough to MDMA to be able to replace MDMA as an adjunct to therapy, but they are not cross-tolerant so that individuals who no longer react appropriately to MDMA can use 5-MAPB or 6-APB as a substitute.

According to an aspect of some embodiments of the present invention, the 5-MAPB or 6-APB is administered prior to the psychotherapeutic intervention.

According to an aspect of some embodiments of the present invention, the 5-MAPB or 6-APB is administered during the psychotherapeutic intervention.

According to an aspect of some embodiments of the present invention, the 5-MAPB or 6-APB is administered at least twice during the psychotherapeutic intervention.

According to an aspect of some embodiments of the present invention, the 5-MAPB or 6-APB is administered after the psychotherapeutic intervention.

According to an aspect of some embodiments of the present invention, the 5-MAPB or 6-APB is administered to the subject in an amount of approximately 50 mg to approximately 500 mg per therapeutic intervention.

According to an aspect of some embodiments of the present invention, the 5-MAPB or 6-APB is administered to the subject in an amount of approximately 100 mg to approximately 400 mg per therapeutic intervention.

According to an aspect of some embodiments of the present invention, the 5-MAPB or 6-APB is administered to the subject in an amount of approximately 100 mg to approximately 200 mg per therapeutic intervention.

According to an aspect of some embodiments of the present invention, the 5-MAPB or 6-APB is administered to the subject in an amount of approximately 100 mg or 200 mg per therapeutic intervention.

According to an aspect of some embodiments of the present invention, the psychotherapeutic intervention is for distress of the subject.

According to an aspect of some embodiments of the present invention, treating is for reducing the distress in the subject.

According to an aspect of some embodiments of the present invention, the distress is selected from the group consisting of cognitive, emotional, behavioral, relationship and spiritual distress.

According to an aspect of some embodiments of the present invention the distress is a condition or disorder selected from the group consisting of personal history of psychological trauma, parent-child relational problem, personal history (past history) of neglect in childhood, personal history (past history) of physical abuse in childhood, personal history (past history) of psychological abuse in childhood, personal history (past history) of sexual abuse in childhood, personal history (past history) of spouse or partner neglect, personal history (past history) of spouse or partner psychological abuse, personal history (past history) of spouse or partner physical violence, personal history (past history) of spouse or partner sexual violence, phase of life problem, relationship distress with spouse or intimate partner, stimulant use or other use disorders and posttraumatic stress disorder (PTSD).

According to an aspect of some embodiments of the present invention the subject is diagnosed with a stress-sensitive disorder selected from the group consisting of Post-traumatic Stress Disorder (PTSD), Bipolar Disorder, Acute Stress Disorder, anxiety disorders such as Generalized Anxiety Disorder, Obsessive-Compulsive Disorder, social anxiety disorders, Panic Disorders, phobias, obsessive compulsive disorders, addictive disorders and Trichotillomania. According to an aspect of some embodiments of the present invention, the psychotherapeutic intervention is selected from the group consisting of psychoanalytic and psychodynamic therapy, behavioral therapy, cognitive therapy, humanistic therapy and integrative or holistic therapy.

According to an aspect of some embodiments of the present invention, the psychotherapeutic intervention is humanistic therapy or integrative/holistic therapy.

According to an aspect of some embodiments of the present invention, reducing the distress comprises at least one of the following: improving stability in the work/home environment, improving behaviors, improving employment functioning, improving vocational functioning, reducing cognitive stress, reducing emotional stress, improving the subject's living situation, improving the subject's family relationships, improving social relationships, improving the subject's sense of meaning to life, improving the subject's sense of purpose to life, reducing mental health symptoms, reducing addiction behaviors and reducing chemical dependencies.

According to an aspect of some embodiments of the present invention, reducing the distress comprises reducing the number and/or frequency of psychotherapeutic interventions, and/or reducing addiction behaviors and reducing chemical dependencies.

According to an aspect of some embodiments of the present invention there is provided a kit for use in treating a subject undergoing a psychotherapeutic intervention comprising a therapeutically effective amount of 5-MAPB or 6-APB and instructions for the use of 5-MAPB or 6-APB in conjunction with the psychotherapeutic intervention.

According to an aspect of some embodiments of the present invention, the psychotherapeutic intervention is for distress in the subject.

According to an aspect of some embodiments of the present invention, the psychotherapeutic intervention is selected from the group consisting of psychoanalytic and psychodynamic therapy, behavioral therapy, cognitive therapy, humanistic therapy and integrative or holistic therapy.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### DESCRIPTION OF SPECIFIC EMBODIMENTS OF THE INVENTION

The present invention relates to methods of drug-assisted psychotherapy with 5-MAPB or 6-APB in a subject for whom MDMA-assisted psychotherapy is either ineffective, undesirable, or inappropriate. The present invention also relates to kits and/or medication combinations comprising 5-MAPB or 6-APB for use in the treatment of subjects undergoing psychotherapeutic intervention.

Drug-assisted psychotherapy combines the psychostimulant properties of certain psychoactive drugs with a structured, psychotherapeutic environment to provide heightened insight, flexibility, trust and patient-therapist rapport. Clinical trials of drug-assisted psychotherapy with psychostimulants such as MDMA have suggested beneficial effects when used in the psychotherapeutic context. However, sometimes MDMA is not effective.

The present inventor has shown that 5-MAPB or 6-APB provides significant enhancement of therapeutic efficacy when used in the psychotherapeutic context for individuals for whom MDMA therapy is ineffective. Surprisingly, the present inventor has shown that even a relatively limited regimen of 5-MAPB or 6-APB-assisted psychotherapy sessions is sufficient to overcome significant obstacles to the patient's progress in therapy. Inter alia, 5-MAPB or 6-APB-assisted psychotherapy is effective in post-traumatic stress disorder (PTSD) therapy, couple's therapy, and therapy for anxiety disorders.

One criterion in the success of psychotherapy or counselling is the connection between the therapist and the patient. The present inventor has found that 5-MAPB or 6-APB can promote empathy and help to build a “therapeutic alliance” with the therapist, which can be an important predictor of a positive outcome for therapeutic or counselling process, encouraging deeper self-disclosure and greater follow-up in behavioral change commitments between sessions. 5-MAPB or 6-APB may produce a sense of calming empowerment, allowing the individual to lower barriers preventing change and review, reframe, revise and even eliminate undesirable associations and memory patterns.

Still further, the present inventor has observed that 5-MAPB or 6-APB is effective in the psychotherapeutic context in part due to its “portal effect,” or deeply felt sense of accomplishment and transition, as well as 5-MAPB or 6-APB's stimulation of creativity and relaxation of rigidity of thought patterns.

Thus, according to some embodiments, the present invention provides a method for treating a subject undergoing a psychotherapeutic intervention comprising administering to said subject 5-MAPB or 6-APB.

The term “treating” means inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein the phrase “psychotherapeutic intervention” means an intervention by a therapist employing methods of psychotherapy, with the intent of modifying an outcome. As used herein, the term “psychotherapy” means the use of psychological, as opposed to medical, methods, particularly when based on regular personal interaction with a therapist, with the goal of helping a person change and overcome problems in desired ways.

The realm of psychotherapeutic interventions includes a great variety of treatment modalities, ranging from traditional Freudian psychoanalysis to holistic therapies. Thus, according to some embodiments, psychotherapeutic interventions suitable for use with the methods of the invention include, but are not limited to psychoanalytic and psychodynamic therapy, behavioral therapy, cognitive therapy, humanistic therapy and integrative or holistic therapy.

Different categories of psychotherapeutic interventions can be distinguished from one another by their approach to well being. For example, as used herein, psychoanalytic therapy means a therapy based on Sigmund Freud's theories of the conscious and unconscious mind. Methods included within psychoanalytic therapy include, but are not limited to psychoanalysis, Freudian psychotherapy, abreaction therapy and transactional analysis.

As used herein, “psychodynamic therapy” means a type of psychology which has the primary focus of resolving unconscious conflicts in the human psyche. Interventions included within psychodynamic therapy include, but are not limited to Adlerian psychotherapy, Jungian psychotherapy, dreamwork therapy, intensive short-term dynamic psychotherapy, primal therapy and process-oriented therapy.

As used herein, “behavioral therapy” means a type of therapy based on the belief that behaviors are learned and that unhealthy behaviors can be changed. Interventions included within behavioral therapy include, but are not limited to behavioral therapy, dialectical behavioral therapy, prolonged exposure therapy, flooding therapy, multimodal therapy, brief psychotherapy, functional analytic psychotherapy, reality therapy and systematic desensitization therapy.

As used herein, “cognitive therapy” means a type of therapy based on the belief that thinking patterns can be problematic and maladaptive thought processes can be changed. Interventions included within cognitive therapy include, but are not limited to, cognitive therapy, cognitive behavioral therapy, rational emotive behavioral therapy, emotional regulation therapy, stress reduction therapy, multimodal therapy, brief psychotherapy, rational living therapy and reality therapy.

As used herein, “humanistic therapy” means a type of therapy which looks at the whole person and believes that people are innately good and want to grow and self-actualize. Interventions included within humanistic therapy include, but are not limited to, humanistic therapy, gestalt therapy, client centered therapy, compassion focused therapy, existential therapy, emotionally focused therapy, emotional freedom technique therapy, motivational interviewing, Rogerian psychotherapy, attachment-based psychotherapy, dyadic developmental psychotherapy, narrative therapy and positive psychology therapy.

As used herein, “integrative” or “holistic therapy” means a mixture of a variety of therapy techniques which includes all aspects of humanity and therefore is directed to an individual's overall physical, mental, spiritual, and emotional wellbeing. Interventions included within integrative or holistic therapy include, but are not limited to integrative therapy, holistic therapy, art therapy, expressive therapy, integrative body therapy, integral therapy, guided imagery therapy, visualization therapy, breathwork therapy, holotropic breathwork therapy, hypnotherapy, psychodrama therapy, eye movement desensitization and reprocessing therapy, sexual identity therapy, solution focused brief therapy, somatic therapy, somatic experiencing therapy, somatic psychology, systemic therapy, thought field analysis, mindfulness meditation therapy, addiction treatment therapy and transtheoretical model of change therapy.

It will be appreciated that any of the abovementioned psychotherapeutic interventions suitable for use with the methods of the invention can be employed individually with 5-MAPB or 6-APB administration, as well as two or more types of psychotherapeutic interventions used in combination. In a non-limiting example, psychotherapeutic interventions suitable for use in the methods of the present invention can include cognitive-behavioral therapy combined with eye movement desensitization and reprocessing therapy (EMDR), or psychoanalysis combined with stress-reduction therapy.

Psychotherapeutic interventions particularly suited for use with the method of the invention include, but are not limited to interventions employing one or more of the following interventional elements: innovation rather than direction, non-directive compassion, encouraging subjects inner healing intelligence, paraphrasing, minimal encouragement, verbal and non-verbal, reflecting, emotional labeling, validating, allowing participants to arrive at their own conclusions, reassurance and waiting, unconditional positive regard, attunement and following not leading.

In some embodiments, a therapeutically effective amount of 5-MAPB or 6-APB is administered to the subject. In some embodiments, 5-MAPB or 6-APB is administered to the subject in an amount in the range of 5 mg per therapeutic intervention to 1000 mg per therapeutic intervention. In some embodiments, 5-MAPB or 6-APB is administered to the subject in an amount in a range of approximately 7 mg to approximately 950 mg per therapeutic intervention, approximately 7 mg to approximately 950 mg per therapeutic intervention, approximately 10 mg to approximately 900 mg per therapeutic intervention, approximately 15 mg to approximately 850 mg per therapeutic intervention, approximately 20 mg to approximately 800 mg per therapeutic intervention, approximately 25 mg to approximately 780 mg per therapeutic intervention, approximately 30 mg to approximately 750 mg per therapeutic intervention, approximately 35 mg to approximately 700 mg per therapeutic intervention, approximately 40 mg to approximately 650 mg per therapeutic intervention, approximately 45 mg to approximately 600 mg per therapeutic intervention, approximately 50 mg to approximately 550 mg per therapeutic intervention, approximately 52 mg to approximately 500 mg per therapeutic intervention, approximately 57 mg to approximately 480 mg per therapeutic intervention, approximately 60 mg to approximately 450 mg per therapeutic intervention, approximately 63 mg to approximately 430 mg per therapeutic intervention, approximately 70 mg to approximately 400 mg per therapeutic intervention, approximately 75 mg to approximately 370 mg per therapeutic intervention, approximately 77 mg to approximately 350 mg per therapeutic intervention, approximately 80 mg to approximately 330 mg per therapeutic intervention, approximately 85 mg to approximately 300 mg per therapeutic intervention, approximately 90 mg to approximately 280 mg per therapeutic intervention, approximately 95 mg to approximately 250 mg per therapeutic intervention, approximately 100 mg to approximately 230 mg per therapeutic intervention, approximately 105 mg to approximately 200 mg per therapeutic intervention, approximately 110 mg to approximately 190 mg per therapeutic intervention, approximately 115 mg to approximately 175 mg per therapeutic intervention, approximately 120 mg to approximately 165 mg per therapeutic intervention, approximately 125 mg to approximately 160 mg per therapeutic intervention or approximately 130 mg to approximately 155 mg per therapeutic intervention. In some embodiments, 5-MAPB or 6-APB is administered to the subject in an amount in a range of 20 mg to 500 mg, 22 mg to 490 mg, 27 mg to 470 mg, 30 mg to 450 mg, 33 mg to 435 mg, 40 mg to 410 mg, 47 mg to 390 mg, 50 mg to 375 mg, 55 mg to 360 mg, 57 mg to 350 mg, 60 mg to 320 mg, 63 mg to 300 mg, 67 mg to 290 mg, 70 mg to 270 mg, 75 mg to 260 mg, 77 mg to 250 mg, 80 mg to 240 mg, 85 mg to 220 mg, 90 mg to 210 mg or 100 mg to 200 mg per therapeutic intervention. It will be appreciated that each individual range of amount of 5-MAPB or 6-APB administered represents a single, separate embodiment.

In some embodiments, 5-MAPB or 6-APB is administered to the subject in an amount of 20, 25, 30, 35, 40, 45, 50, 55, 60, 65, 67, 70, 73, 75, 77, 80, 83, 85, 87, 90, 93, 95, 97, 100, 103, 107, 110, 113, 115, 117, 120, 123, 125, 127, 130, 133, 137, 140, 143, 145, 147, 150, 153, 155, 157, 160, 163, 167, 170, 173, 175, 177, 180, 183, 185, 187, 190, 193, 195, 197, 200, 220, 240, 250, 275, 290, 300, 310, 320, 330, 340, 350, 360, 370, 380, 390 or 400 mg per therapeutic intervention. In particular embodiments, 5-MAPB or 6-APB is administered in an amount of 50 mg per therapeutic intervention, 100 mg per therapeutic intervention, 150 mg per therapeutic intervention, 200 mg per therapeutic intervention, 250 mg per therapeutic intervention, 300 mg per therapeutic intervention, 350 mg per therapeutic intervention or 400 mg per therapeutic intervention.

In some embodiments, 5-MAPB or 6-APB is administered to the subject in a dosage calculated according to mass per body weight, e.g., mg 5-MAPB or 6-APB per Kg body weight of the subject. In some embodiments, 5-MAPB or 6-APB is administered to the subject in a dosage range of approximately 0.5 mg/Kg to approximately 7 mg/Kg body weight per therapeutic intervention, approximately 0.75 mg/Kg to approximately 6.5 mg/Kg body weight per therapeutic intervention, approximately 1.0 mg/Kg to approximately 6.0 mg/Kg body weight per therapeutic intervention, approximately 1.25 mg/Kg to approximately 5.5 mg/Kg body weight per therapeutic intervention, approximately 1.5 mg/Kg to approximately 5 mg/Kg body weight per therapeutic intervention, approximately 1.75 mg/Kg to approximately 4.5 mg/Kg body weight per therapeutic intervention, approximately 2.0 mg/Kg to approximately 4.25 mg/Kg body weight per therapeutic intervention, approximately 2.25 mg/Kg to approximately 4.0 mg/Kg body weight per therapeutic intervention, approximately 2.5 mg/Kg to approximately 3.75 mg/Kg body weight per therapeutic intervention, approximately 2.75 mg/Kg to approximately 3.5 mg/Kg body weight per therapeutic intervention body weight per therapeutic intervention. It will be appreciated that each individual range of 5-MAPB or 6-APB per Kg body weight administered represents a single, separate embodiment.

In some embodiments, 5-MAPB or 6-APB is administered to the subject in a dosage of 1.0, 1.25, 1.5, 1.75, 2.0, 2.25, 2.5, 2.75, 3.0, 3.25, 3.5, 3.75, 4.0, 4.25, 4.5, 4.75, 5.0 or 5.5 mg/Kg body weight per therapeutic intervention. In specific embodiments, 5-MAPB or 6-APB is administered to the subject in a dosage of 1, 1.5, 2, 2.5 or 3 mg/Kg body weight per therapeutic intervention.

5-MAPB or 6-APB can be administered to the subject via methods including, but not limited to oral administration, intravenous administration, insufflation (nasal administration), mucosal administration, rectal administration, transdermal administration and inhalation (e.g. dry powder or vapor inhalation). In specific embodiments of the method of the invention, the 5-MAPB or 6-APB is administered to the subject via oral administration. In particular embodiments, 5-MAPB or 6-APB is administered orally to the subject in an amount of 50 mg per therapeutic intervention, 100 mg per therapeutic intervention, 150 mg per therapeutic intervention, 200 mg per therapeutic intervention, 250 mg per therapeutic intervention, 300 mg per therapeutic intervention, 350 mg per therapeutic intervention or 400 mg per therapeutic intervention.

It will be appreciated that a psychotherapeutic intervention can be an individual, or isolated psychotherapeutic intervention, e.g., a therapy or counseling “session”, or a part of a regimen of psychotherapeutic intervention, for example, a series of such psychotherapeutic intervention “sessions”.

As used herein the phrase “treatment regimen” means a treatment plan that specifies the type of treatment, dosage, schedule and/or duration of a treatment provided to a subject in need thereof (e.g., a subject diagnosed with a pathology, condition or disorder). The selected treatment regimen can be an aggressive one which is expected to result in the best clinical outcome (e.g., complete cure of the pathology) or a more moderate one which may relief symptoms of the pathology yet results in incomplete cure of the pathology. It will be appreciated that in certain cases the more aggressive treatment regimen may be associated with some discomfort to the subject or adverse side effects (e.g., nausea, tachycardia). The dosage, schedule and duration of treatment can vary, depending on the severity of condition/disorder/pathology and the selected type of treatment, and those of skills in the art are capable of adjusting the type of treatment with the dosage, schedule and duration of treatment.

In some embodiments of the method of the invention, the 5-MAPB or 6-APB is administered once during the psychotherapeutic intervention, or once per session. In other embodiments, the 5-MAPB or 6-APB is administered more than once during the psychotherapeutic intervention, e.g., twice during the psychotherapeutic intervention or session, three times during the psychotherapeutic intervention or session, four times during the psychotherapeutic intervention or session or more than four times during the psychotherapeutic intervention or session.

In some embodiments, the psychotherapeutic intervention is effected prior to, concomitant with, or shortly after exposure to 5-MAPB or 6-APB. The time interval between the psychotherapeutic intervention and administering the 5-MAPB or 6-APB, or between administering the 5-MAPB or 6-APB and the psychotherapeutic intervention can range from 1 hour to 48 hours, from 1 hour to 24 hours, from 1 hour to 18 hours, from 1 hour to 16 hours, from 1 hour to 12 hours, from 1 hour to 10 hours, from 1 hour to 8 hours, from 1 hour to 6 hours, from 1 hour to 4 hours and from 1 hour to 2 hours, 0.1 hours to 2 hours, from 0.25 hours to 1.5 hours and from 0.5 hours to 1.0 hours. The desired time interval can be determined according to, for example, the formulation of the 5-MAPB or 6-APB, the condition to be treated, the treated subject.

Safety and pharmacokinetic studies with animal models (Shimshoni et al J Psychopharm, 2015; 29:734-43) have indicated that peak serum concentrations of 5-MAPB or 6-APB are apparent within 5-10 minutes after oral administration, and that the apparent plasma half-life, after oral ingestion, is approximately 1 hour. The present inventor has observed that, in some embodiments the onset of human subject's perception of 5-MAPB or 6-APB's effect is approximately 30-90 minutes following oral administration, varying according to whether the 5-MAPB or 6-APB was administered to a fasting (more rapid onset), or following a meal (less rapid onset). In some cases, the perception of peak effect of 5-MAPB or 6-APB in human subjects occurred between 10 to 30 minutes after the initial perception of onset of 5-MAPB or 6-APB's effect. The present inventors further observed that the duration of perception of the effect of 5-MAPB or 6-APB in human subjects was, in most cases, for no greater than 4 hours following oral administration.

Thus, in some embodiments, the 5-MAPB or 6-APB is administered at least twice during the psychotherapeutic intervention or session. In some embodiments, the 5-MAPB or 6-APB is administered twice during the psychotherapeutic intervention or session, at intervals of 1-8 hours between dosings. In other embodiments, the 5-MAPB or 6-APB is administered twice or three times during the psychotherapeutic intervention or session, at intervals of 2-7 hours between dosings. In other embodiments, the 5-MAPB or 6-APB is administered twice or three times during the psychotherapeutic intervention or session, at intervals of 3-6 hours between dosings.

In other embodiments, the 5-MAPB or 6-APB is administered twice or three times during the psychotherapeutic intervention or session, at intervals of 1-4 hours between dosings. In other embodiments, the 5-MAPB or 6-APB is administered twice or three times during the psychotherapeutic intervention or session, at intervals of 1-3 hours between dosings. In yet other embodiments, the 5-MAPB or 6-APB is administered twice or more during the psychotherapeutic intervention or session, at intervals of 1-2 hours between dosings,

In some embodiments, 5-MAPB or 6-APB is administered in multiple doses (e.g. more than once) during the psychotherapeutic intervention or session, where each administration is of the same dosage of 5-MAPB or 6-APB (e.g. 3×100 mg, 2×150 mg, 2×200 mg, and the like). In other embodiments, 5-MAPB or 6-APB is administered in multiple doses (e.g. more than once) during a psychotherapeutic intervention or session, where each administration is of a different dosage of 5-MAPB or 6-APB (e.g. 1×150 mg followed by 1×100 mg; 1×200 mg followed by 1×100 mg, followed by a third dose of 1×100 mg; 1×200 mg, followed by 1×150 mg, and the like). Combinations of initial higher and subsequent lower doses, as well as combinations of initial lower, and subsequent higher doses, as well as subsequent dosages alternating between lower and higher than the initial dose are also envisioned.

It will be appreciated that, in some embodiments, psychotherapeutic interventions comprising administration of 5-MAPB or 6-APB, according to the methods of the present invention, can be consecutive interventions comprising administration of 5-MAPB or 6-APB (e.g. sessions) within a regimen or treatment plan of psychotherapeutic interventions, and in other embodiments, interventions comprising administration of 5-MAPB or 6-APB can be interspersed within a regimen or treatment plan which comprises psychotherapeutic interventions without administration of 5-MAPB or 6-APB. In some embodiments in which interventions comprising administration of 5-MAPB or 6-APB are interspersed within a regimen or treatment plan which comprises psychotherapeutic interventions without administration of 5-MAPB or 6-APB, the psychotherapeutic interventions comprising administration of 5-MAPB or 6-APB can be interspersed regularly between interventions without administration of 5-MAPB or 6-APB throughout the regimen or treatment plan, or, in other embodiments, psychotherapeutic interventions comprising administration of 5-MAPB or 6-APB can be included between with interventions without administration of 5-MAPB or 6-APB in a non-regular manner, throughout the regimen or treatment plan. In one non-limiting example, a series of psychotherapeutic interventions (sessions) comprising administration of 5-MAPB or 6-APB may be included for a limited period of time as an adjunct to a treatment plan or regimen predominantly including interventions without administration of 5-MAPB or 6-APB. Such a regimen or treatment plan can be implemented, for example, in order to address a specific aspect of a subject's therapy especially suited for treatment with the psychotherapeutic interventions comprising 5-MAPB or 6-APB of the present invention.

According to some embodiments, a regimen or treatment plan comprising psychotherapeutic interventions (sessions) of the method of the present invention comprising administration of 5-MAPB or 6-APB can include psychotherapeutic interventions (sessions) of the method of the present invention at intervals of 2-4 hours, 4-6 hours, 6-12 hours, 12-18 hours, 18-24 hours, 36 hours, 48 hours, 3 days, 4 days, 5 days, 6 days, 1 week, 2 weeks, 3 weeks, 1 month, 2 months, 3 months or more. As detailed hereinabove, a regimen or treatment plan comprising psychotherapeutic interventions (sessions) of the method of the present invention comprising administration of 5-MAPB or 6-APB can include psychotherapeutic interventions (sessions) of the method of the present invention at regular intervals during a regimen of treatment, or irregularly throughout a regimen.

As used herein, the subject is a mammalian subject, and preferably a human subject. Psychotherapeutic interventions are often directed to modifying an outcome, and/or helping a person change and/or overcome problems in desired ways. In some embodiments, subject is suffering from distress, and the psychotherapeutic intervention of the method of the present invention is for distress of the subject. In specific embodiments, the treating the subject with the method of the present invention is for reducing distress of the subject suffering from distress.

As used herein, the term “distress” means reaction to a “stressor” and constituting an underlying component of anxiety and depression. Distress can lead to a disorder that results in altered or abnormal behavior, function, or subjective distress in one or more of the following intrapersonal or interpersonal realms: mood, anxiety, memory, cognition, consciousness, perception, sexual experience, sleep, substance use/addiction, personality, attention/concentration, psychosis, identity, eating, or bodily function or integrity.

In some embodiments, the subject is suffering from distress selected from the group consisting of cognitive distress, emotional distress, behavioral distress, relationship distress and spiritual distress.

As used herein, cognitive distress means distress which results from problematic and/or inaccurate thinking processes, for example, someone who justifies abusive behavior, and takes no responsibility and offers the rationalization that the other person is to blame.

As used herein, emotional distress means distress which is related to feelings. Examples are problematic anger, fear, or grief.

As used herein, behavioral distress means distress that is observable to others as it is manifested in a person's behavior, for example, aggression to others or self-harm by self-mutilation.

As used herein, relationship distress means distress which results from conflict in a relationship. One non-limiting example is of couple in the process of a conflictual divorce.

As used herein, spiritual distress means distress related to the larger issue(s) of meaning and purpose to life. One non-limiting example is an individual who attempts suicide and then has a spiritual revelation that they need to reach out to others who are suicidal.

Typically, a subject suffering from distress experiences social, interpersonal, and/or occupational impairment or dysfunction. The cause (etiology, or stressor) may be idiopathic (unknown), or it may be due to a recognized psychosocial stressor, a medical disorder, or a neurological disorder. It should be appreciated that any type distress can be compatible with aspects of the invention.

5-MAPB or 6-APB-assisted psychotherapeutic intervention according to the method of the present invention can be suitable for a wide range of disorders and conditions which are indicated for psychotherapeutic intervention. Many such diagnostic categories are listed in the American Psychiatric Society's Diagnostic and Statistical Manual, Fifth Edition (DSM-V, 2013) and in the World Health Organization's International Statistical Classification of Diseases and Related Health Problems, 10^{th} Revision (ICD 10), in particular, Chapter V of the ICD 10: “Mental and Behavioral Disorders”. Thus, according to some embodiments of the invention, the subject may be diagnosed with a condition or disorder selected from the group consisting of the disorders and conditions in following Table I:

TABLE I

| | Disorder, | Disorder, |
|---|---|---|
| | Condition or | Condition or |
| Disorder, Condition or | Problem | Problem |
| Problem - Designation | DSM-V | ICD-10 |
| Acute stress disorder | 308.3 | F43.0 |
| Adjustment disorder | | |
| Adjustment disorder, Unspecified | 309.9 | F43.20 |
| Adjustment disorder, With anxiety | 309.24 | F43.22 |
| Adjustment disorder, With depressed mood | 309.0 | F43.21 |
| Adjustment disorder, With disturbance | 309.3 | F43.24 |
| of conduct | | |
| Adjustment disorder, With mixed | 309.28 | F43.23 |
| anxiety and depressed mood | | |
| Adjustment disorder, With mixed disturbance | 309.4 | F43.25 |
| of emotions and conduct | | |
| Adult physical abuse by nonspouse or | | |
| nonpartner, Confirmed | | |
| Adult sexual abuse by nonspouse or | | |
| nonpartner, Confirmed | | |
| Agoraphobia | 300.22 | F40.00 |
| Amphetamine-type substance use disorder | | |
| Anorexia nervosa | 307.1 | |
| Anorexia nervosa, Binge-eating/purging type | | F50.02 |
| Anorexia nervosa, Restricting type | | F50.01 |
| Antisocial personality disorder | 301.7 | F60.2 |
| Anxiety disorder due to another | 293.84 | F06.4 |
| medical condition | | |
| Attention-deficit/hyperactivity disorder | | |
| Avoidant personality disorder | 301.82 | F60.6 |
| Avoidant/restrictive food intake disorder | 307.59 | F50.8 |
| Binge-eating disorder | 307.51 | F50.8 |
| Body dysmorphic disorder | 300.7 | F45.22 |
| Child sexual abuse, Suspected or Confirmed | | |
| Cocaine use disorder | | |
| Cocaine use disorder-Mild | 305.60 | F14.10 |
| Cocaine use disorder-Moderate | 304.20 | F14.20 |
| Cocaine use disorder-Severe | 304.2 | F14.20 |
| Conduct disorder | | |
| Dependent personality disorder | 301.6 | F60.7 |
| Depressive disorder due to another | 293.83 | |
| medical condition | | |
| Depressive disorder due to another | | F06.31 |
| medical condition, With depressive | | |
| features | | |
| Depressive disorder due to another | | F06.32 |
| medical condition, With major | | |
| depressive-like episode | | |
| Depressive disorder due to another | | F06.34 |
| medical condition, With mixed features | | |
| Discord with neighbor, lodger, or landlord | V60.89 | Z64.4 |
| Disruption of family by separation or divorce | V61.03 | Z63.5 |
| Female orgasmic disorder | 302.73 | F52.31 |
| Female sexual interest/arousal disorder | 302.72 | F52.22 |
| Gambling disorder | 312.31 | F63.0 |
| Generalized anxiety disorder | 300.02 | F41.1 |
| High expressed emotion level within family | V61.8 | Z63.8 |
| Hoarding disorder | 300.3 | F42 |
| Illness anxiety disorder | 300.7 | F45.21 |
| Kleptomania | 312.32 | F63.2 |
| Major depressive disorder, Recurrent episode | | |
| Male hypoactive sexual desire disorder | 302.71 | F52.0 |
| Narcissistic personality disorder | 301.81 | F60.81 |
| Obsessive-compulsive disorder | 300.3 | F42 |
| Obsessive-compulsive personality disorder | 301.4 | F60.5 |
| Overweight or obesity | | |
| Other circumstances related to adult | | |
| abuse by nonspouse or nonpartner | | |
| Other circumstances related to spouse | | |
| or partner neglect | | |
| Other circumstances related to spouse | | |
| or partner violence, physical | | |
| or sexual | | |
| Other circumstances related to spouse | | |
| or partner abuse, Psychological | | |
| Other circumstances related to child neglect | | |
| Other circumstances related to child | | |
| psychological abuse | | |
| Other circumstances related to child | | |
| sexual abuse | | |
| Other specified sexual dysfunction | | |
| Other specified personality disorder | 301.89 | F60.89 |
| Other specified anxiety disorder | 300.09 | F41.8 |
| Other specified feeding or eating disorder | 307.59 | F50.8 |
| Other specified gender dysphoria | 302.6 | F64.8 |
| Other specified trauma- and stressor- | 309.89 | F43.8 |
| related disorder | | |
| Other (or unknown) substance use disorder | | |
| Other or unspecified stimulant use disorder | | |
| Other personal history of psychological | V15.49 | Z65.8 |
| trauma | | |
| Opioid use disorder | | |
| Oppositional-defiant disorder | 313.81 | F91.3 |
| Parent-child relational problem | V61.20 | Z62.820 |
| Personal history (past history) of | V15.42 | Z62.812 |
| neglect in childhood | | |
| Personal history (past history) of | V15.41 | Z62.810 |
| physical abuse in childhood | | |
| Personal history (past history) of | V15.42 | Z62.811 |
| psychological abuse in childhood | | |
| Personal history (past history) | V15.41 | Z62.810 |
| of sexual abuse in childhood | | |
| Personal history (past history) | V15.42 | Z91.412 |
| of spouse or partner neglect | | |
| Personal history (past history) of | V15.42 | Z91.411 |
| spouse or partner psychological abuse | | |
| Personal history (past history) of | V15.41 | Z91.410 |
| spouse or partner violence, physical | | |
| Personal history (past history) of | V15.41 | Z91.410 |
| spouse or partner violence, sexual | | |
| Personal history of self-harm | V15.59 | Z91.5 |
| Phase of life problem | V62.89 | Z60.0 |
| Posttraumatic stress disorder | 309.81 | F43.10 |
| Reactive attachment disorder | 313.89 | F94.3 |
| Relationship distress with spouse | V61.10 | Z63 |
| or intimate partner | | |
| Religious or spiritual problem | V62.89 | Z65.8 |
| Sedative, hypnotic, or anxiolytic | | |
| use disorder | | |
| Separation anxiety disorder | 309.21 | F93.0 |
| Sex counseling | V65.49 | Z70.9 |
| Sibling relational problem | V61.8 | Z62.891 |
| Social anxiety disorder (social phobia) | 300.23 | F40.10 |
| Social exclusion or rejection | V62.4 | Z60.4 |
| Spouse or partner abuse, | | |
| Psychological, Confirmed | | |
| Stimulant use disorder | | |
| Tobacco use disorder | | |
| Uncomplicated bereavement | | |
| Unspecified anxiety disorder | 300.00 | F41.9 |
| Unspecified alcohol-related disorder | 291.9 | F10.99 |
| Unspecified caffeine-related disorder | 292.9 | F15.99 |
| Unspecified cannabis-related disorder | 292.9 | F12.99 |
| Unspecified cocaine-related disorder | | F14.99 |
| Unspecified communication disorder | 307.9 | F80.9 |
| Unspecified gender dysphoria | 302.6 | F64.9 |
| Unspecified personality disorder | 301.9 | F60.9 |
| Unspecified problem related to | V62.9 | Z65.9 |
| social environment | | |
| Unspecified depressive disorder | 311 | F32.9 |
| Unspecified tobacco-related disorder | 292.9 | F17.209 |
| Victim of crime, terrorism or torture | V62.82 | Z65.4 |

In specific embodiments, the subject is diagnosed with a condition or disorder selected from the group consisting of personal history of psychological trauma, parent-child relational problem, personal history (past history) of neglect in childhood, personal history (past history) of physical abuse in childhood, personal history (past history) of psychological abuse in childhood, personal history (past history) of sexual abuse in childhood, personal history (past history) of spouse or partner neglect, personal history (past history) of spouse or partner psychological abuse, personal history (past history) of spouse or partner physical violence, personal history (past history) of spouse or partner sexual violence, phase of life problem, relationship distress with spouse or intimate partner, stimulant use disorder and posttraumatic stress disorder.

In specific embodiments, the subject is diagnosed with relationship distress with spouse or intimate partner, religious or spiritual problem, stimulant use disorder, post-traumatic stress disorder or tobacco use disorder.

In some embodiments, the subject is diagnosed with a stress-sensitive disorder. As used herein, a “stress-sensitive disorder” means any condition, disease or disorder that results, at least in part, from exposure to stress or is exacerbated, at least in part, from exposure to stress. Non-limiting examples of stress-sensitive disorders include Post-traumatic Stress Disorder (PTSD), Bipolar Disorder, Acute Stress Disorder, anxiety disorders such as Generalized Anxiety Disorder, Obsessive-Compulsive Disorder, social anxiety disorders, Panic Disorders, phobias, obsessive compulsive disorders, and Trichotillomania. It should be appreciated that any stress-sensitive disorder can be compatible with aspects of the invention.

It will be appreciated that, in some embodiments, the subject may be diagnosed with more than a single condition or disorder. In the event of multiple diagnoses for a subject's condition, psychotherapeutic interventions and/or the methods of treating of the present invention may be directed to treatment of a single diagnosis among the multiple diagnoses, according to priorities determined by the therapist and/or subject, or, in some embodiments, it may be suitable to treat more than a single condition or disorder among the subject's diagnoses concomitantly, rather than sequentially. For example, and just by way of illustration, in a subject diagnosed with both substance addiction and depression, the therapist's treatment regimen may choose to include use of the method(s) of the invention for treatment of the depression before proceeding to treatment of the substance addiction, or the therapist may choose to include use of the method(s) of the invention in treatment of some of the multiple diagnoses of the subject, and use other therapeutic modalities in addressing the subject's multiple diagnoses.

Administering 5-MAPB or 6-APB to a subject undergoing psychotherapeutic intervention according to the methods of the present invention can be useful in reducing distress in a subject in need thereof. In some embodiments, reducing the distress comprises identifying and changing thought and/or behavior patterns in said subject.

Typically, the goal of psychotherapeutic intervention is to help the subject change and overcome problems in desired ways. One of the outcomes of effective psychotherapeutic intervention is a reduction in the number and/or frequency of interventions required for particular conditions/diagnoses. Further non-limiting outcomes of effective psychotherapeutic intervention include, but are not limited to greater stability in the work/home environment, improved behaviors, improved employment functioning, improved vocational functioning, reduction of cognitive stress, reduction of emotional stress, improved living situation, improved family relationships, improved social relationships, improved sense of meaning to life, improved sense of purpose to life, reduction of mental health symptoms, reduction of addiction behaviors, reduction in chemical dependencies and the like.

Thus, in some embodiments, where the subject is being treated with the method of the present invention for reducing distress, reducing distress in the subject comprises reducing the number and/or frequency of psychotherapeutic interventions. In other embodiments, reducing distress in the subject comprises improving stability in the work/home environment, improving behaviors, improving employment functioning, improving vocational functioning, reducing cognitive stress, reducing emotional stress, improving the subject's living situation, improving the subject's family relationships, improving social relationships, improving the subject's sense of meaning to life, improving the subject's sense of purpose to life, reducing mental health symptoms, reducing addiction behaviors and reducing chemical dependencies. In specific embodiments, reducing distress in the subject comprises one or more of reducing chemical dependencies, greater vocational or academic functioning and improved family and/or social relationships.

Methods and instruments for diagnosing, evaluating the severity of and monitoring the progress of therapy of a subject's psychological or psychiatric disorder or condition being treated with psychotherapeutic intervention are well known in the art.

A non-limiting list of suitable instruments for diagnosing, monitoring and evaluating subjects' progress prior to, during or following therapy using the methods of the present invention is provided in Table II:

TABLE II
MONITORING AND OUTCOME MEASURES

| Objective | Measure | Link/Info |
|---|---|---|
| Safety | Adverse Events | |
| | Serious Adverse | |
| | Events | |
| | Monitor Session | Griffiths et al., 2006 |
| | Rating Form | |
| | Challenging | Used in Josh Woolley UCSF protocol and |
| | Experience | by the Johns Hopkins Group. |
| | Questionnaire | The Challenging Experience Questionnaire: |
| | | Characterization of challenging experiences |
| | | with psilocybin mushrooms. |
| | | Barrett et al, J Psychopharmacol 2016; |
| | | 12: 1279-9 |
| Feasibility | Rate of patient | |
| | recruitment | |
| | Rate of patient | |
| | retention | |
| | Client Satisfaction | Larsen et al 1979 Evaluation and Program |
| | Questionnaire 8 | Planning 2: 197-207 |
| | Qualitative: Patient | British Columbia Centre on Substance |
| | Experience | Abuse |
| Efficacy - | Timeline Follow-back | Robinson et al 2014 Psych Addict Behav |
| Decreased | (NIDA) | 28: 154-62 |
| substance use | Urine Drug Screen | |
| | Urinary Cotinine Test | |
| | (tobacco) | |
| | Carbon Monoxide | |
| | Breath Test (tobacco) | |
| | Hair/Nail Analysis | |
| | (Alcohol) | |
| | DSM-5 Substance | bup practice node/19556 |
| | Use Disorder | |
| | Diagnostic Checklist | |
| | Addiction Severity | m. breining |
| | Index-Lite (ASI-Lite) | |
| | Severity of | Adapted from Gossop et al 1995 Addiction |
| | Dependence Scale | 90: 607-614 |
| | Structured Clinical | See Am Psychiatric Assoc website |
| | Interview for DSM-5 | |
| | Research Version | |
| | (SCID-5-RV)- | |
| | Module E: Substance | |
| | Use Disorders | |
| | Short Inventory of | Example found is alcohol only version, but |
| | Problems - Alcohol | Alcohol and Drug version does exist. See |
| | and Drugs | CASAA in University of New Mexico .edu |
| | | website |
| Efficacy - | Thoughts About | Hall S M, Havassy B E, Wasserman D A. |
| Enhanced | Abstinence | Commitment to abstinence and acute stress |
| motivation to | Questionnaire | in relapse to alcohol, opiates, and nicotine. |
| reduce or stop | | J Consult Clin Psychol. 1990; 58: 175-181. |
| substance use | | The four-item TAA was used to assess |
| | | motivation to quit and abstinence self- |
| | | efficacy. TAA motivation to quit scores |
| | | have been shown to predict smoking |
| | | cessation treatment outcome, and TAA |
| | | abstinence self-efficacy scores have been |
| | | shown to mediate the effect of extended |
| | | cognitive behavioral therapy on treatment |
| | | outcome |
| | Substance Use | See Kings College (UK) website- Scales, |
| | Recovery Evaluator | Measures and Instruments |
| | Self Efficacy Scales | See University of Maryland, Baltimore |
| | | County website Self-Efficacy Scales |
| | Readiness To Change | Treatment Improvement Protocol (TIP) |
| | Questionnaire | Series, No. 35. |
| | (Treatment Version) | Center for Substance Abuse Treatment. |
| | RCQ-TV | Rockville (MD): Substance Abuse and |
| | | Mental Health Services Administration |
| | | (US); 1999. |
| | Recovery Progression | Initial Development and Psychometric |
| | Measure | Properties of a New Measure of Substance |
| | | Use Disorder “Recovery Progression”: The |
| | | Recovery Progression Measure (RPM) |
| | | Elison S, Davies G, Ward J. |
| | | Subst Use Misuse. 2016 Jul. 28; 51(9): 1195- |
| | | 206 |
| | Drug-Taking | Annis, H. M., Sklar, S. M. & Turner, N. E. |
| | Confidence | (1997). The Drug-Taking Confidence |
| | Questionnaire | Questionnaire (DTCQ): User's Guide. |
| | | Toronto, Canada: Addiction Research |
| | | Foundation, Centre for Addiction and |
| | | Mental Health. Annis, H. M. & Martin, G. |
| | | (1985). Drug-Taking Confidence |
| | | Questionnaire. Toronto, Canada: Addiction |
| | | Research Foundation. |
| | The Stages of Change | CASAA in University of New Mexico .edu |
| | Readiness and | website |
| | Treatment Eagerness | |
| | Scale (SOCRATES | |
| | 8A) | |
| | Alcohol Abstinence | DiClemente, C. C., Carbonari, J. P., |
| | Self-Efficacy Scale | Montgomery, R. P. G. & Hughes, S. O. |
| | | (1994). The Alcohol Abstinence Self- |
| | | Efficacy Scale. Journal of Studies on |
| | | Alcohol, 55, 141-148. |
| Mechanism - | Mystical Experience | R R Griffiths, R. R., W A Richards, W. A., U |
| Evaluate | Questionnaire | McCann, U., & R Jesse, R., (2006), |
| whether or not | | “Psilocybin can occasion mystical-type |
| characteristics | | experiences having substantial and |
| of the | | sustained personal meaning and spiritual |
| psilocybin | | significance”. Psychopharmacology, |
| administration | | 187(3), 268-83; commentaries on pp. 284- |
| session | | 292. |
| experiences | Altered State of | Dittrich, A, Lamparter, D, Maurer, M. |
| (e.g. | Consciousness | Zurich, Switzerland: PSIN PLUS; 2006. |
| Intensity) | Questionnaire (5D- | 5D-ABZ: Fragebogen zur Erfassung |
| mediate | ASC) | Aussergewohnlicher Bewusstseinszustande. |
| effects of | | Eine kurze Einfuhrung [5D-ASC: |
| psilocybin on | | Questionnaire for the assessment of altered |
| short-term | | states of consciousness. A short |
| persisting | | introduction]. |
| effects and | Persisting Effects | Griffiths, R. R., Richards, W. A., McCann, |
| post-session | Questionnaire | U., & Jesse, R. (2006), “Psilocybin can |
| substance use | | occasion mystical-type experiences having |
| behaviour | | substantial and sustained personal meaning |
| | | and spiritual significance.” |
| | | Psychopharmacology, 187(3), 268-83, |
| | | commentaries on pp. 284-292. |
| | States of | Griffiths, R. R., Richards, W. A., McCann, |
| | Consciousness | U., & Jesse, R. (2006), “Psilocybin can |
| | Questionnaire | occasion mystical-type experiences having |
| | | substantial and sustained personal meaning |
| | | and spiritual significance.” |
| | | Psychopharmacology, 187(3), 268-83, |
| | | commentaries on pp. 284-292. |
| | Hallucinogen | Could not locate sample online. Used in |
| | Rating Scale | Bogenschutz protocol and by Griffiths |
| | | et al 2006. |
| | Mysticism Scale | See Northern Virginia Community College |
| | | website: Laura Shulman |
| | APZ | Used in Hendricks protocol and by |
| | | Griffiths et al 2006. Based on article below, |
| | | the 5D-ASC could be an updated version. |
| | | Psychometric Evaluation of the Altered |
| | | States of Consciousness Rating Scale |
| | | (OAV) |
| | | Erich Studerus, * Alex Gamma, and Franz |
| | | X. Vollenweider PLoS One. 2010; 5(8): |
| | | e12412. |
| | The Addiction | Could not find example online. Used by |
| | Research Center | Bogenschutz et al 2015 and by Griffiths |
| | Inventory (ARCI), 49- | et al 2006 |
| | item version | |
| | Visual Effects | Used by Johnson et al 2014. |
| | Questionnaire | |
| Neurobio | fMRI Craving Task | What other fMRI tasks might be useful to |
| Mechanism | Pre- & Post- | shed light on potential mechanisms & |
| | experimental session | lasting effects of psilocybin on neural |
| | | activity? |
| | | Stress response, Response to emotional |
| | | stimuli, Inhibitory control tasks? |
| | | Task-free fMRI assessing resting state |
| | | functional connectivity? |
| Efficacy - | | |
| Mediators of | | |
| treatment | | |
| Motivation | Thoughts About | see above |
| | Abstinence | |
| | Questionnaire | |
| | Substance Use | see above |
| | Recovery Evaluator | |
| | Readiness To Change | see above |
| | Questionnaire | |
| | (Treatment Version) | |
| | RCQ-TV | |
| | Readiness Ruler | See ADULT MEDUCATION website |
| | | Assessment Tools |
| Self-efficacy | Self Efficacy Scales | see above |
| | Drug-Taking | see above |
| | Confidence | |
| | Questionnaire | |
| Craving | Brief Substance | Somoza, E., Dyrenforth, S., Goldsmith, J., |
| | Craving Scale | Mezinskis, J., & Cohen, M., 1995. In |
| | | search of a universal drug craving scale. |
| | | Paper presented at the Annual Meeting of |
| | | the American Psychiatric Association, |
| | | Miami Florida. |
| | | See Univ of Washington website: ADAI |
| | Alcohol Craving | |
| | Visual Analog Scale | |
| | (ACVAS) | |
| | Short Inventory of | Miller et al, National Institute on Alcohol |
| | Problems (SIP) Scale | Abuse and Alcoholism Project MATCH |
| | | Monograph Series Volume 4 Pages 49, 51 |
| Depression | PROMIS Emotional | 2008 PROMIS Health Organization and |
| | Distress - Depression | PROMIS Cooperative Group |
| | (used by VIDUS) | |
| | Centre for | National Institutes of Mental Health, US |
| | Epidemiologic | |
| | Studies Depression | |
| | Scale | |
| | Beck's Depression | |
| | Inventory | |
| | Hamilton Depression | |
| | Rating Scale | |
| | Quick Inventory of | Rush et al, Biol Psychiatry (2003) 54: 573- |
| | Depressive | 83. |
| | Symptomology | |
| Anxiety | PROMIS Emotional | PROMIS Item Bank v1.0 - Emotional |
| | Distress - Anxiety | Distress - Anxiety - Short Form 8a |
| | (used by VIDUS) | |
| | State-Trait Anxiety | Speilberger et al. Mind Garden Redwood |
| | Inventory | CA |
| | Beck's Anxiety | Beck, A. T., Epstein, N., Brown, G., Steer, |
| | Inventory | R. A. (1988). An inventory for measuring |
| | | clinical anxiety: Psychometric properties. |
| | | Journal of Consulting and Clinical |
| | | Psychology, 56, 893-897. |
| | Hamilton Anxiety | Hamilton M. The assessment of anxiety |
| | Rating Scale | states by rating. Br J Med Psychol 1959; |
| | | 32: 50-55. |
| Mood | Profile of Mood States | Top End Sports and |
| | Questionnaire | McNair et al. (1971) Manual for the Profile |
| | | of Mood States. San Diego, CA: |
| | | Educational and Industrial Testing Service. |
| | | Grove, J. R., & Prapavessis, H. (1992). |
| | | Preliminary evidence for the reliability and |
| | | validity of an abbreviated Profile of Mood |
| | | States. International Journal of Sport |
| | | Psychology, 23, 93-109. |
| Self- | Self-Compassion | 2011, Raes et al, Clin Psych Psychother 18 |
| Compassion | Scale (Short Form) | 250-255 |
| Spiritual | Persisting Effects | See above |
| Dimensions | Questionnaire | (also has items on depression & anxiety) |
| | Spirituality Index | Daaleman, T. P. & Frey, B. B. (2004). The |
| | of Well Being | Spirituality Index of Well-Being: A new |
| | | instrument for health-related quality of life |
| | | research. Annals of Family Medicine, 2, |
| | | 499-503. |
| | Strength of Religious | Used in Hendricks protocol. |
| | Faith Questionnaire | |
| | Meaning in Life | Steger, M. F., Frazier, P., Oishi, S., & |
| | Questionnaire | Kaler, M. (2006). The Meaning in Life |
| | | Questionnaire: Assessing the presence of |
| | | and search for meaning in life. Journal of |
| | | Counseling Psychology, 53, 80-93. |
| | Satisfaction with | Diener, E., Emmons, R. A., Larsen, R. J., & |
| | Life Scale | Griffin, S. (1985). The Satisfaction with |
| | | Life Scale. Journal of Personality |
| | | Assessment, 49, 71-75 |
| | ASPIRES Spiritual | See Bogenschutz protocol, by Bogenschutz |
| | Transcendence Scale | et al 2015, and by Griffiths et al 2006. |
| | Brief | See: Fetzer Institute, National Institute on |
| | Multidimensional | Aging Working Group: Multidimensional |
| | Measure of | Measurement of Religiousness, Spirituality |
| | Religiousness/ | for Use in Health Research. A Report of a |
| | Spirituality | National Working Group. Supported by the |
| | | Fetzer Institute in Collaboration with the |
| | | National Institute on Aging. Kalamazoo, |
| | | MI: Fetzer Institute, 2003 (1999) and |
| | | Research on Aging: “Measuring Multiple |
| | | Dimensions of Religion and Spirituality for |
| | | Health Research,” Ellen L. Idler, Marc A. |
| | | Musick, Christopher G. Ellison, Linda K. |
| | | George, Neal Krause, Marcia G. Ory, |
| | | Kenneth I. Pargament, Lynda H. Powell, |
| | | Lynn G. Underwood, David R. Williams, |
| | | 2003, 25: 4. |

In specific embodiments, a subject's progress prior to, during, or following therapy using the methods of the present invention is evaluated using at least one of the Beck Depression Inventory, the Beck Anxiety Inventory, the Beck Hopelessness Scale, for PTSD: UCLA PTSD Index for DSM IV, GAD-7, The Primary Care PTSD Screen and others, the Oxford Happiness Inventory and Addictions assessments selected from Table 2.

It will be appreciated that the method of the present invention can be combined with other therapeutic modalities, in addition to the psychotherapeutic intervention. Pharmacotherapy is a common adjunct to psychotherapeutic intervention in modern treatment plans, for achieving both short term and long-term results in the subject undergoing psychotherapeutic intervention. Thus, in some embodiments, the subject is being treated with at least one drug selected from the group consisting of selective serotonin re-uptake inhibitors (SSRIs), mono-amine-oxidase (MAO) inhibitors, serotonin-dopamine antagonists, analgesics, antihypertensive drugs, anti-allergenics, anti-inflammatory drugs, poison antidotes, anti-convulsive drugs, anti-infective drugs, muscle relaxants and local anesthetics. In specific embodiments, the method of the present invention further comprises administering to the subject at least one drug selected from above mentioned group.

Yet further, non-pharmacological adjuncts to psychotherapy have become popular recently, and can also be integrated within the psychotherapeutic intervention of the methods of the present invention. In addition, the context of the psychotherapy is important and attention needs to be paid to both the set (ie expectations of the client/patient/subject) and the setting or environment of the experience (eg music, art, decorations, food, videos, etc.). Thus, in some embodiments, the method of the present invention further comprises exposing the subject to an adjunct therapeutic, non-pharmacological modality selected from the group consisting of, for example, at least one of music, food, choice of space for therapy, visual stimulus, audio stimulus, thermal comfort, art, books, interior design, objects that have an impact (e.g. spiritual cards), presence (or absence) of other individuals, therapist mannerisms, furnishings suitable for therapy sessions.

The 5-MAPB or 6-APB of some embodiments of the invention can be administered to a subject per se, or in a pharmaceutical composition where it is mixed with suitable carriers or excipients.

As used herein a “pharmaceutical composition” means a preparation of one or more of the active ingredients described herein with other chemical components such as physiologically suitable carriers and excipients. The purpose of a pharmaceutical composition is to facilitate administration of a compound to an organism.

Herein the term “active ingredient” means the 5-MAPB or 6-APB accountable for the biological effect.

Hereinafter, the phrases “physiologically acceptable carrier” and “pharmaceutically acceptable carrier” which may be interchangeably used, refer to a carrier or a diluent that does not cause significant irritation to an organism and does not abrogate the biological activity and properties of the administered compound. An adjuvant is included under these phrases.

Herein the term “excipient” means an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols. Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide.

In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of 5-MAPB or 6-APB effective, when combined with psychotherapeutic intervention, to treat, prevent, alleviate or ameliorate symptoms of a disorder or condition in the subject.

Determination of a therapeutically effective amount is well within the capability of those skilled in the art, especially in light of the detailed disclosure provided herein.

For any preparation used in the methods of the invention, the therapeutically effective amount or dose can be estimated initially from animal models. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide levels of 5-MAPB or 6-APB sufficient to induce or suppress the biological effect minimal effective concentration, (MEC). The MEC will vary for each preparation, but can be estimated from animal models. Dosages necessary to achieve the MEC will depend on individual characteristics and route of administration. Detection assays can be used to determine plasma concentrations. As detailed hereinabove, depending on the severity and responsiveness of the condition to be treated, dosing can be of a single or a plurality of administrations, with course of treatment lasting from several days to several weeks or until cure is effected or diminution of the disorder or condition is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing 5-MAPB or 6-APB as the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

Thus, according to some embodiments, there is provided a kit or combination with other medicines or ingredients for use in treating a subject undergoing a psychotherapeutic intervention comprising a therapeutically effective amount of 5-MAPB or 6-APB and instructions for the use of 5-MAPB or 6-APB in conjunction with said psychotherapeutic intervention. Types of psychotherapeutic interventions suitable for combination with administering 5-MAPB or 6-APB according to the methods of the present invention are described in detail hereinabove. In some embodiments, the instructions indicate that the psychotherapeutic intervention is for distress in the subject. In other embodiments, the psychotherapeutic intervention is selected from the group consisting of psychoanalytic and psychodynamic therapy, behavioral therapy, cognitive therapy, humanistic therapy and integrative or holistic therapy.

It is expected that during the life of a patent maturing from this application many relevant methods for treating subjects undergoing psychotherapeutic intervention with 5-MAPB or 6-APB will be developed and the scope of the term “administering 5-MAPB or 6-APB to a subject undergoing a psychotherapeutic intervention” is intended to include all such new technologies a priori.

As used herein the term “about” or “approximately” means±10%.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” means manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

What is claimed is:

1. A method for treating a subject undergoing a psychotherapeutic intervention comprising administering to said subject 5-MAPB or 6-APB, wherein the subject is resistant to treatment with MDMA.

2. The method of claim 1, wherein said 5-MAPB or 6-APB is administered prior to said psychotherapeutic intervention.

3. The method of claims 1, wherein said 5-MAPB or 6-APB is administered during said psychotherapeutic intervention.

4. The method of claim 3, wherein said 5-MAPB or 6-APB is administered at least twice during said psychotherapeutic intervention.

5. The method of claim 1, wherein said 5-MAPB or 6-APB is administered after said psychotherapeutic intervention.

6. The method of any one of claims 1-5, wherein said 5-MAPB or 6-APB is administered to said subject in an amount of approximately 50 mg to approximately 500 mg per therapeutic intervention.

7. The method of claim 6, wherein said 5-MAPB or 6-APB is administered to said subject in an amount of approximately 100 mg to approximately 400 mg per therapeutic intervention.

8. The method of claim 6, wherein said 5-MAPB or 6-APB is administered to said subject in an amount of approximately 100 mg to approximately 200 mg per therapeutic intervention.

9. The method of any one of claims 1-8, wherein said 5-MAPB or 6-APB is administered to said subject in an amount of approximately 100 mg or 200 mg per therapeutic intervention.

10. The method of any one of claims 1-9, wherein said psychotherapeutic intervention is for distress of said subject.

11. The method of claim 10, wherein said treating is for reducing said distress in said subject.

12. The method of claim 10 or 11, wherein said distress is selected from the group consisting of cognitive, emotional, behavioral, relationship and spiritual distress.

13. The method of any one of claims 10-12, wherein said distress is a condition or disorder selected from the group consisting of personal history of psychological trauma, parent-child relational problem, personal history (past history) of neglect in childhood, personal history (past history) of physical abuse in childhood, personal history (past history) of psychological abuse in childhood, personal history (past history) of sexual abuse in childhood, personal history (past history) of spouse or partner neglect, personal history (past history) of spouse or partner psychological abuse, personal history (past history) of spouse or partner physical violence, personal history (past history) of spouse or partner sexual violence, phase of life problem, relationship distress with spouse or intimate partner, stimulant use disorder and posttraumatic stress disorder.

14. The method of any one of claims 10-12, wherein said subject is diagnosed with a stress-sensitive disorder selected from the group consisting of Post-traumatic Stress Disorder (PTSD), Bipolar Disorder, Acute Stress Disorder, anxiety disorders such as Generalized Anxiety Disorder, Obsessive-Compulsive Disorder, social anxiety disorders, Panic Disorders, phobias, obsessive compulsive disorders, and Trichotillomania.

15. The method of any one of claims 1-14, wherein said psychotherapeutic intervention is selected from the group consisting of psychoanalytic and psychodynamic therapy, behavioral therapy, cognitive therapy, humanistic therapy and integrative or holistic therapy.

16. The method of claim 15, wherein said psychotherapeutic intervention is humanistic therapy or integrative/holistic therapy.

17. The method of claim 10, wherein reducing said distress comprises at least one of the following: improving stability in the work/home environment, improving behaviors, improving employment functioning, improving vocational functioning, reducing cognitive stress, reducing emotional stress, improving the subject's living situation, improving the subject's family relationships, improving social relationships, improving the subject's sense of meaning to life, improving the subject's sense of purpose to life, reducing mental health symptoms, reducing addiction behaviors and reducing chemical dependencies.

18. The method of claim 10, wherein reducing said distress comprises reducing the number and/or frequency of psychotherapeutic interventions, and/or reducing addiction behaviors and reducing chemical dependencies.

19. A kit for use in treating a subject undergoing a psychotherapeutic intervention comprising a therapeutically effective amount of 5-MAPB or 6-APB and instructions for the use of 5-MAPB or 6-APB in conjunction with said psychotherapeutic intervention.

20. The kit of claim 19, wherein said psychotherapeutic intervention is for distress in said subject.

21. The kit of claim 19 or 20, wherein said psychotherapeutic intervention is selected from the group consisting of psychoanalytic and psychodynamic therapy, behavioral therapy, cognitive therapy, humanistic therapy and integrative or holistic therapy.
